(19) United States
(12) Patent Application Publication
(10) Pub. No.: US 2026/0251579 A1
(43) Pub. Date: Aug. 27, 2026

(54) CONTROL OF THE INJECTION OF A LIQUID ONTO A TEST SUPPORT

(71) Applicant: WITHINGS, ISSY LES MOULINEAUX (FR)

(72) Inventors: Antoine MERLOT, ISSY LES MOULINEAUX (FR); Brice BRAC DE LA PERRIÈRE, ISSY LES MOULINEAUX (FR)

(21) Appl. No.: 19/544,523

(22) Filed: Feb. 19, 2026

(30) Foreign Application Priority Data
Feb. 20, 2025 (FR) FR2501746

(51) Int. Cl. G01N 21/78; G01N 21/25; G01N 35/10

(52) U.S. Cl. CPC G01N 21/78; G01N 21/251; G01N 35/1016; G01N 2035/1025

(57) ABSTRACT

An injection control method for injecting a liquid onto a test support, the test support comprising an absorbent material capable of absorbing the liquid and a reagent capable of reacting with at least one component present in the liquid, the method being implemented using an injector and an optical sensor.

### CROSS-REFERENCE TO RELATED APPLICATIONS

This application claims priority to French Patent Application No. FR2501746, filed February 20, 2025, the entire content of which is incorporated herein by reference in its entirety

### FIELD

The present invention relates to the automatic analysis of test supports, in particular for testing bodily fluids (e.g., urine). The automatic analysis can be implemented in a "home" urine analysis device, also known as a "point of care" device, rather than on laboratory or hospital equipment. The test supports generally take the form of strips.

### BACKGROUND

When analyzing a test support, it is desirable to know whether the strip has been properly saturated with the relevant fluid (urine, blood, saliva, etc.). For so-called "lateral flow" strips (known as immunochromatographic strips), control lines are generally provided. Document EP2385369 uses an optical detector to identify the control line that may appear. However, for colorimetric strips that incorporate colorimetric pads, control lines are not necessary, particularly because the colorimetric pad is often directly soaked by the liquid deposited on it. In a lateral flow test strip, the movement of a liquid front causes a reaction in a test zone and in a control zone beyond the test zone, where the control line is located. On a traditional colorimetric test support, there is no movement of urine.

However, for automation purposes, it is preferable to have a device that works equally well on colorimetric test supports and lateral flow test supports. Similarly, for a personal device that can be used at home, it is preferable to have a compact and robust device. However, these devices may present difficulties related to the flow rate of an injector, which may be variable, or difficulties related to the movement of certain test supports inside the device, or difficulties related to the aging of the test supports (color change, etc.).

### SUMMARY

This description therefore proposes an automatic injection control method for controlling the injection of a liquid onto a test support using an injector and an optical sensor.

According to one aspect, the description proposes an injection control method for controlling the injection of a liquid onto a test support, the test support comprising an absorbent material adapted to absorb the liquid and a reagent adapted to react with at least one component present in the liquid, the method being implemented using an injector and an optical sensor, the method comprising:

injecting the liquid onto the absorbent material of the test support by the injector,

calculating a change in a physical property value (the change being related to the injection of liquid onto the absorbent material) using the optical sensor of a control region of the test support during injection,

in response to determining that the variation is below a threshold, ending the injection, and/or

in response to determining that the variation is greater than a threshold (the same threshold), continuing to inject liquid onto the test support.

In an embodiment, the calculation of the variation is performed using at least two values of the physical property taken at time intervals with liquid injection between the two measurements. In particular, the method may include obtaining a value relating to the physical property before injection.

In an embodiment, the injection is performed in intermittent injection sequences and the variation in the value is calculated from at least one value obtained before an injection sequence and at least one value obtained after the injection sequence.

The value obtained before an injection sequence may be obtained by averaging several values obtained before injection.

In general, the average of several values may be a temporal average (over several successive images) and/or a spatial average (over the entire control region or a portion thereof).

In this embodiment, a maximum number of injection sequences may be predefined.

In one example, at least three injection sequences take place and at least two variation calculations are performed before the end of the injection.

In an embodiment, the injection is continuous and the variation calculation is performed continuously.

In an embodiment of the test support, the absorbent material defines a liquid pathwayfrom an injection region, which receives liquid from the injector, to the control region, where the reagents are located, along the pathway between the injection region and the control region.

In an embodiment, the control region is a region of the absorbent material, for example an extremal region located beyond the reagents along the liquid pathway, and the value is relative to the color of the control region. Once saturated with liquid, the color of the control region changes slightly.

In an embodiment, the control region is a colorimetric pad on the test support. The physical property may be the color of the colorimetric pad. Alternatively or additionally, the physical property is a dimension of the colorimetric pad. This is because the pad expands as it becomes saturated.

The method may further comprise, in response to the end of the injection, analysis of the test support using the optical sensor.

According to one aspect, the description also relates to a test support analysis station, comprising an injector and an optical sensor, the analysis station being configured to implement a method as described above.

In an embodiment, the optical sensor is configured to view the entire test support, i.e., at least one side of the test support.

According to one aspect, the description also relates to a urine analysis device comprising a urine analysis station as described above and a test support, wherein the test support comprises an absorbent material adapted to absorb liquid and a reagent adapted to react with at least one component present in the liquid.

According to one aspect, the description relates to a non-transitory computer program product (e.g. a non-transitory computer readable medium) comprising instructions adapted to implement a method as described above when the instructions are executed by a processor, in particular a processor of a station as described.

According to an embodiment, there is provided a method of controlling delivery of urine to a test support in a urine analysis system, the method comprising: causing an injector to deliver urine to a test support that includes an absorbent material; obtaining, using an optical sensor, a plurality of optical measurements associated with a control region of the test support at different times during said delivery; determining, by control circuitry based on the plurality of optical measurements, successive values of a monitored metric representative of a physical property of the control region; computing, by the control circuitry, a change value indicative of an amount of change of the monitored metric between the successive values; comparing the change value to a threshold; and controlling the injector based on the comparing, including stopping the delivery of urine in response to determining that the change value is below the threshold, thereby indicating saturation of the control region.

### BRIEF DESCRIPTION OF THE DRAWINGS

Other features, details, and benefits will become apparent upon reading the detailed description below and analyzing the accompanying drawings, in which:

FIG. 1 presents a schematic and simplified representation of a urine analysis device installed in a toilet bowl,

FIG. 2 shows an exploded view of the urine analysis device, in which the station and the cartridge are visible,

FIG. 3 shows a schematic representation of a fluidic circuit of a measuring station,

FIG. 4 shows a cross-sectional view of a cartridge and a station according to one embodiment, at the location of an optical analyzer of the station.

FIG. 5 shows an embodiment of a test support, with the injection end illustrated and the field of view of an optical sensor represented,

FIG. 6 shows the evolution over time of a test support schematically (A) and in reality (B).

FIG. 7 shows a diagram of a method according to an embodiment of the invention, and

FIG. 8 shows a schematic architecture of a measuring station and its ecosystem.

### DETAILED DESCRIPTION

This description presents an injection control method for injecting a liquid (e.g., urine) onto test supports. This method can be implemented using a station comprising an injector and an optical sensor. The station typically receives the test supports using a cartridge that houses a plurality of them to form a test device. In an embodiment, the device is sized to be placed on the bowl wall of a toilet. The following documents describe an example of such an analysis device: WO2021175909 and WO2021175944, WO2023036805, WO2023036806, WO2023036808, WO2023036809. Hereinafter, these documents will be referred to as WO documents for general reference.

### Description of the station and the analysis device

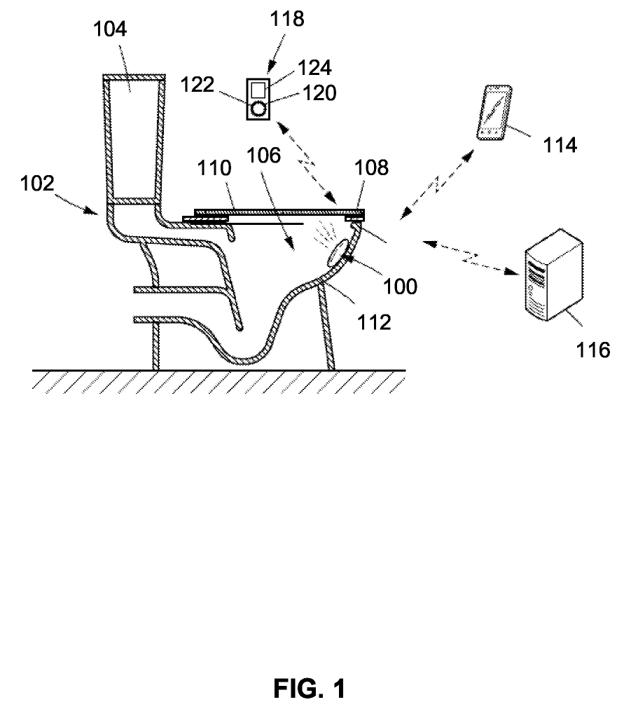

FIG. 1 schematically illustrates a urine analysis device 100 (hereinafter also referred to as "device 100") for analyzing urine installed in toilet 102. The toilet 102 generally comprises a water tank 104, a bowl 106, a seat 108, and a seat cover 110. The analysis device 100 is configured to be placed entirely within the toilet bowl, but it may be positioned elsewhere. "In the bowl" means "placed within the interior volume defined by the bowl." The analysis device 100 is removably disposed in the toilet 102. For example, the analysis device 100 can be easily removed from the toilet to replace a cartridge and then replaced in the toilet 102. The analysis device 100 is placed on a bowl wall 112 of the toilet bowl 106. The analysis device 100 is positioned such that it is generally below the stream of urine from a user, so that when a user urinates (generally in a seated position), the urine comes into contact with the analysis device 100. The analysis device 100 may communicate remotely with a remote entity, such as the smartphone 114 or a server 116.

However, alternatively, the station may be arranged outside the toilet.

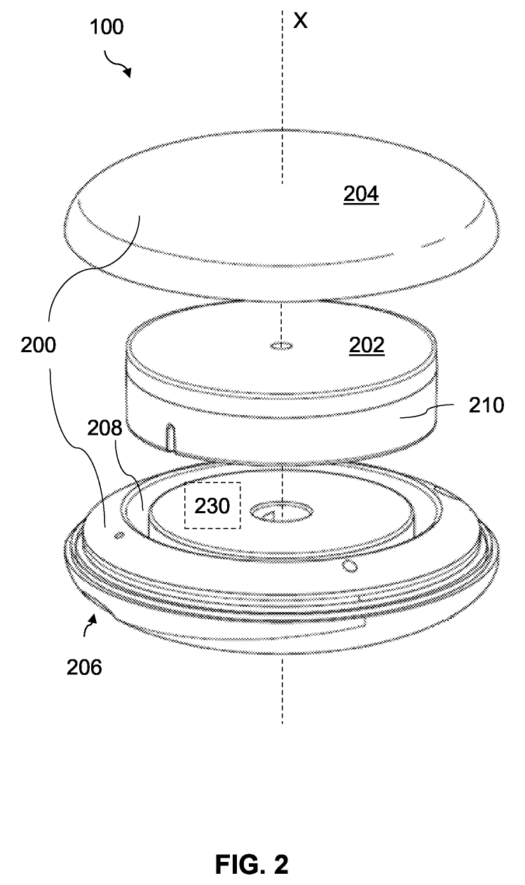

As illustrated in detail in FIG. 2, the urine analysis device 100 comprises a urine analysis station 200 (called "station 200" later) and a cartridge 202, mounted removably on the urine analysis station 200. The cartridge 202 comprises a reagent adapted to react with urine (hereinafter referred to as "urine reagent"). In a cartridge-less embodiment, the urine analysis device 100 and the urine analysis station 200 are combined.

Alternatively, station 200 directly comprises urine reagent without any removable parts.

More details on this case are provided in the WO documents cited previously.

Station 200 comprises a sealed housing 204, whose function is also to collect urine and convey it to a collection port 206 located on housing 204. In an example installation in which the urine analysis device 100 is supported on the bowl wall 112, the collection port 206 is provided on, and oriented to face, a portion of the housing 204 that is oriented toward (e.g., faces) the bowl wall 112 such that the collection port 206 faces the bowl wall 112 when installed. In this example, a portion of the housing 204 exposed on an opposite side of the housing 204 that faces the interior of the toilet bowl 106 is devoid of a urine collection port (e.g., lacks any urine collection opening), such that urine is collected via the collection port 206 on the wall-facing side of the housing 204.

The housing 204 may have a diameter, measured in the direction orthogonal to the X-axis, of between 50 mm and 150 mm. The housing 204 may have a thickness, measured in the direction of the X-axis, of between 15 mm and 50 mm. Thus, the housing 204 is compact enough to be entirely housed in the toilet bowl. The urine analysis device 100 is discreet. In addition, the housing 204 is large enough to systematically come into contact with the urine received in the bowl. The user can then urinate in the toilet without worrying about the urine analysis device, or alternatively aim roughly.

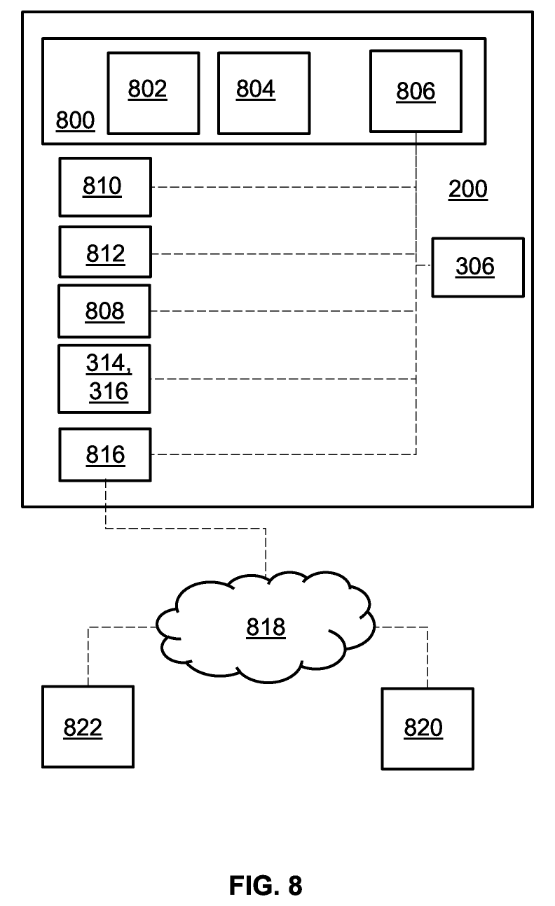

The station 200 includes control circuitry 800, shown in FIG. 8, configured to control the various components of the device 100, such as the position of an injection end or the activation of a pump or, where applicable, a valve, as will be described in detail below.

### Cartridge housing

The station 200 typically comprises an annular compartment 208, located inside the housing 204, arranged around a rotation axis X and configured to receive a cylindrical portion 210 of the cartridge.

In the embodiment shown in the figures, the cartridge 202 comprises urine reagent, in particular by means of a plurality of test supports, each of which comprises at least one urine reagent, for example a dry reagent. In the illustrated example, the plurality of test supports are arranged along a circle or arc of a circle around the axis of rotation X and form the plurality of analysis regions. In an embodiment, the test supports are test strips. The test supports may be enclosed, for example individually, in a sealed chamber.

The annular compartment 208 typically extends over 360° and forms a groove configured to at least partially receive the cartridge 202.

Document EP4338839 describes a method for obtaining sealed chambers in a cartridge.

### Fluidic circuit

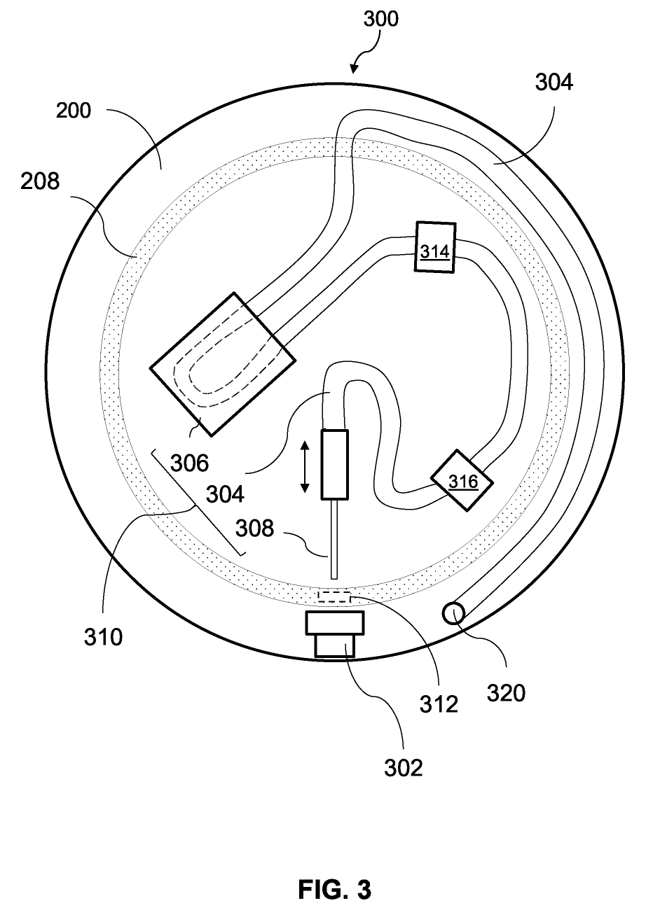

To convey urine that drips onto the housing 204 to the test support, the urine analysis station 200 includes a fluidic circuit 300 illustrated in FIG. 3.

The fluidic circuit comprises a reservoir 302 (e.g., formed at the collection port 206 to collect urine dripping onto the housing 204), piping 304, a pump 306, and an injection end 308. The fluidic circuit may also include a drain port 320 configured to drain liquid from the device 100.

The piping connects the reservoir 302, the pump 306, the injection end 308, and possibly the drain port 320.

In the embodiment illustrated in FIG. 3, the injection end 308 is located at the reservoir 302 (LIFO logic, or "last in, first out"). In a variant not shown, the reservoir 302 may be separated from the injection end 308 (FIFO logic, or first in, first out).

In FIG. 3, a test support 312 is shown (in dotted lines, as FIG. 3 illustrates a station without the cartridge, and is not to scale) to indicate where it is located in relation to the station. The test support 312 has been isolated here from the other test supports and the cartridge.

For more details, see documents WO2023036805, WO2023036806, WO2023036808, WO2023036809, or documents FR2410306, FR2410307, and FR2410308 (filing numbers) filed by Withings in 2024.

In an embodiment, station 200 comprises two fluid presence sensors 314, 316 spaced apart along fluidic circuit 300, thereby defining a reference section (whose predetermined volume is known by control circuitry 800). By measuring the time taken for the fluid to travel between the two fluid presence sensors 314, 316, the control circuitry 800 can calculate the flow rate of the pump 306.

The one or two fluid presence sensors 314, 316 may comprise electrodes or optical probes. Document WO2022184984 describes such sensors (in particular the optical sensor) in detail.

The injection end 308, the pump 306, and the piping 304 form an injector 310. The injection end 308 may include a needle.

The pump 306 may have different types of operation that will impact the operation of the injector 310. For example, the injector 310 may operate by injection sequence, i.e., the pump is activated sequentially, so that the liquid flow rate of the injector 310 is intermittent (e.g., every microliter, the pump stops). Alternatively, injector 310 may operate continuously, i.e., the pump is activated continuously, so that the liquid flow from injector 310 is uninterrupted.

For this operation, pump 306 may operate in "strokes," particularly when pump 306 is peristaltic. A pump stroke is a brief, controlled activation of pump 306. For example, intermittent pump strokes can generate injection by injection sequence (in particular with the correspondence between a pump stroke and an injection sequence), and continuous pump strokes can generate continuous injection.

The injector 310 is controlled by control circuitry 800, described at the end of this description.

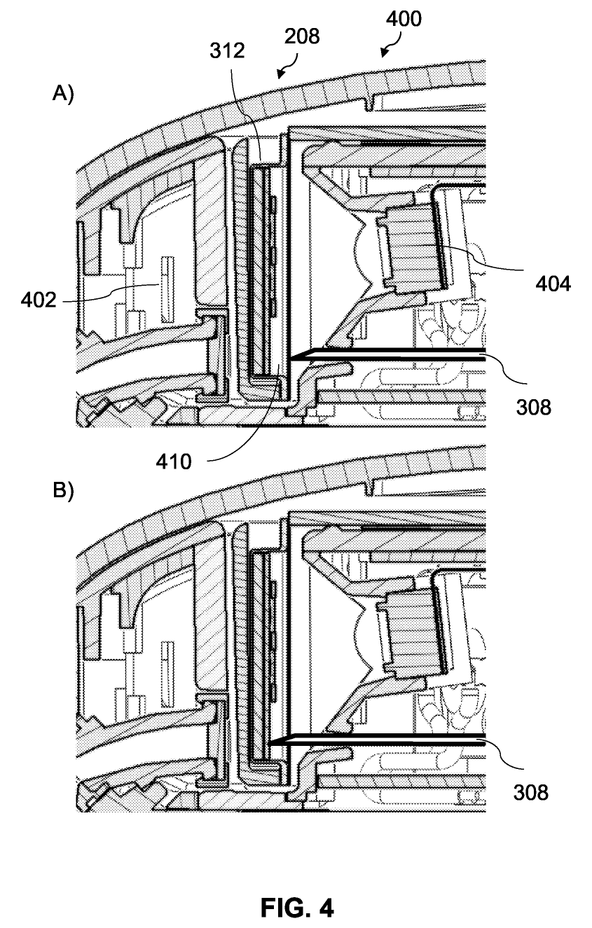

As shown in FIG. 4, which illustrates a cross-sectional view, the injection end 308 can be moved between several positions. In particular, there is a neutral position (FIG. 4A) in which the injection end 308 does not interact with the test support 312 (and therefore does not pass through the sealed chamber 410) and an injection position (FIG. 4B) in which the injection end 308 enters the sealed chamber 410 and comes into contact, or almost into contact, with the test support 312 in order to use the injector to inject urine onto the test support. For example, the injection end 308 is positioned sufficiently close to the test support 312 that a gap therebetween is minimized (e.g., a small standoff distance), such that urine dispensed from the injection end 308 reliably wets a target region of the test support 312 without substantial splashing, misting, or overspray. In some examples, “almost into contact” means the injection end 308 is spaced from the test support 312 by a distance that is less than about 5 mm, less than about 3 mm, or less than about 1 mm, while still avoiding physical contact that could abrade, deform, or contaminate the test support 312.

In the neutral position, the injection end 308 is, in the embodiment illustrated in the figures, located radially inside the sealed chamber 410. This maximizes the radius of the annular compartment while minimizing the size of the station 200.

However, device 100 is only one example of a specific embodiment of an automated urine analysis device.

### The analyzer

The station also includes an analyzer 400, shown in FIG. 4.

The analyzer 400 comprises a light source 402 (e.g., an LED) and at least one optical sensor 404, in this case in the form of a camera, to detect in particular a change in color (e.g., in RGB) or intensity. In the example shown, the light travels from the light source 402 to the optical sensor 404 through the cartridge 202, the test support 312, and thus in particular the urine reagent on the test support 312 (i.e., transmission illumination). Alternatively, the light source may be on the same side as the optical sensor (reflection illumination). Alternatively, the optical sensor 404 measures using ambient light, without a dedicated light source 402.

In an embodiment, the optical sensor 404 can see the entire test support 312 (i.e., one entire side of the test support 312). In other words, the field of view of the optical sensor 404 includes the entire test support 312 (at least one entire side of the test support).

The analyzer 400 is configured to obtain information relating to the urine in the analysis region, whether this information is obtained directly from the urine or indirectly from the urine (via the reagent).

### Test supports

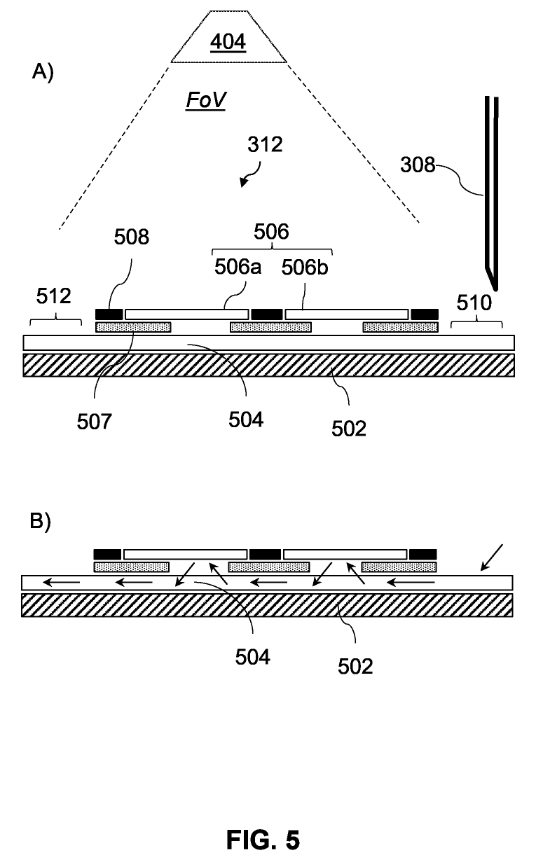

FIG. 5 illustrates one embodiment of test support 312. The test support 312 typically comprises a frame 502 (e.g., a plastic strip to stiffen the test support 312), on which an absorbent material 504 forming a liquid pathway (called a pathway) is shown. The absorbent material may comprise cellulose.

In the embodiment illustrated in FIG. 5, the test support 312 is of the colorimetric type, i.e., it further comprises an analysis region 506, including one or more colorimetric pads 506a, 506b comprising a reagent whose color changes when the pad is brought into contact with a component of the liquid (so that the pad is sensitive to pH, specific gravity, ketones, vitamin C, etc.). The analysis region 506 may be attached to the absorbent material 504 by at least one adhesive 507. One or more masks 508 may be provided to improve optical analysis. These masks and associated variants are described in document EP4339598.

In FIG. 5A), the optical sensor 404, its field of view FoV, and the injection end 308 are shown for clarity (scales are not to scale).

The analysis region 506 is in fluid communication with the absorbent material 504, so that urine can pass from one to the other and vice versa.

FIG. 5 B) illustrates the path of urine on the test support 312. In this regard, the test support 312 includes an injection region 510, configured to be positioned opposite the injection end 308 of the injector 310. The test support 312 further comprises an extremal region 512, located beyond the analysis regions 506 along the flow path.

To saturate the analysis region 506, a sufficient amount of urine is injected into the injection region 510. This is because, due to the configuration of the flow path that carries the liquid to react on the analysis region 506, there is a risk that the liquid will remain on the flow path, favoring passage through the absorbent material 504. Therefore, to ensure that the analysis region 506 has been properly saturated, one option is to saturate the pathway, i.e., the absorbent material 504, with liquid. One of the difficulties is how to determine this saturation in the case of automated liquid injection, i.e., without operator control.

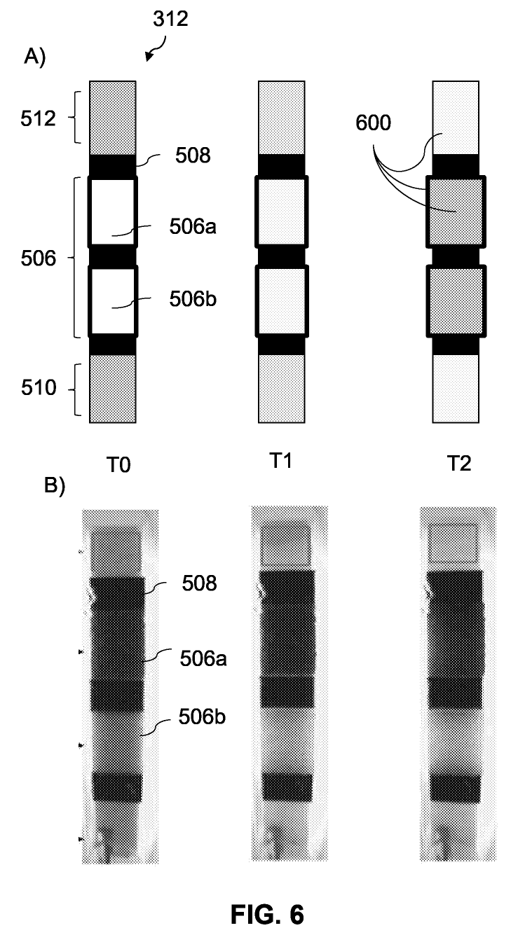

In order to control the injection of liquid onto the test support 312 by the injector 310, the optical sensor 404 analyzes a region of the test support 312, known as the control region 600, several variants of which are illustrated in FIG. 6 and will be explained in detail.

### The injection control method

An automatic injection control method will be described. More specifically, it is an automatic injection control method. In the context described above, its purpose is to ensure that sufficient liquid has been injected. Specifically, in an aspect, the purpose of the automatic injection control method is to provide closed-loop control of the injector 310 to help ensure that a sufficient volume of liquid has been delivered to the test support 312 and, more particularly, that a selected portion of the test support 312 has reached liquid saturation. The principle of this control method is not to detect, via the optical sensor 404, a change in color in a region of the control region following the injection of liquid by the injector 310 (e.g. the appearance of a color change attributable to a dedicated lateral-flow control line), as is the case with lateral flow strips " (where a control line appears to indicate that the liquid has traveled the entire path), but to detect, using the optical sensor 404, the moment when a physical property of the control region 600, which normally changes due to the injection of liquid, ceases to change, despite the injection of liquid. That is, an embodiment of the disclosure detects a transition to steady state—i.e., the time at which a monitored physical property of the control region 600 that normally varies during wetting/imbibition ceases (or substantially ceases) to change even though liquid continues to be injected. This indicates liquid saturation and therefore ensures that the control region 600 has been properly saturated.

FIG. 6 A) shows the temporal evolution of a test support before injection (T0) and during injection (T1 and T2) in the upper part. The temporal order is therefore T0, T1, then T2. FIG. 6 B) shows, for informational purposes only, actual samples, which have been schematized in the upper part.

FIG. 6 illustrates a test support 312 at three different times T0, T1, T2, with the injection starting between T0 and T1. The injector 310 injects liquid onto the injection region 510. The liquid spreads as explained in relation to FIG. 5 B). The control region 600 of the test support 312 may be located at different places depending on the implementation and the particular physical property to be monitored.

In an embodiment, the control region 600 corresponds to the extremal region 512, or to a portion of the extremal region. The control region 600 may be a rectangle. This extremal region 512 is formed by the absorbent material 504. There is no specific reagent in the control region as in the analysis regions, so the extremal region 512 does not contain a reagent for a component of the liquid. However, the absorbent material 504 has physical properties that can change with the absorption of the liquid, such as its transparency and therefore its color. As it becomes saturated with liquid, the absorbent material 504 becomes more transparent, allowing more light to pass through and appearing lighter due to the light source 402. Once saturated with liquid, the transparency of the absorbent material no longer changes and therefore its color no longer changes. The physical property, the value of which is measured by the optical sensor, can therefore be a color (for example, the red channel of the RGB sensor). FIG. 6 A) illustrates a variation in the color of the extremal region 512 at T0, T1, and T2, which becomes lighter. The measured value can be obtained from a spatial average of the control region 600 (or a portion thereof) on an image, but also from a combination of a temporal average over several images (in particular, the temporal average of the spatial averages). In some implementations, the “steady state” condition used to stop injection corresponds to the spatially averaged color value changing by less than a predetermined amount (e.g., less than threshold K) over a predetermined time interval.

In another embodiment, the control region 600 corresponds to the analysis region 506, which is formed by at least one colorimetric pad 506a, 506b. Two variants of this embodiment will be presented.

In a variant of this embodiment, the control region 600 corresponds to the analysis region 506 or to a portion of the analysis region 506. The control region 600 may be a rectangle. The physical property is the color of the analysis region 506. As it becomes saturated, the analysis region 506 reacts with the liquid and changes color. Once the analysis region 506 is saturated with liquid, the color of the colorimetric pad no longer changes. The physical property, the value of which is measured by the optical sensor, can therefore be a color (for example, the red channel of the RGB sensor). The measured value can be obtained from a spatial average of the control region 600 (or a portion thereof) on an image, but also from a combination of a temporal average over several images (in particular the temporal average of the spatial averages). In this variant, injection may be terminated when the rate of change of the measured color value falls below threshold K, indicating that additional injected liquid is no longer producing a material change in the pad’s color response.

In another variant of this embodiment, the physical property is the area of the analysis region 506 and in particular of the colorimetric pad 506a, 506b. As it absorbs liquid, the analysis region 506 becomes saturated and enlarges, resulting in an expansion of the area in a plane parallel to the test support 312 and therefore an increase in its surface area. FIG. 6 A) illustrates the variation in the surface area of the analysis region 506 at T0, T1, and T2, which increases. The optical sensor 404 can measure the number of saturated pixels (due to the light source 402) near the analysis region 506, as this number of saturated pixels decreases as the surface area of the analysis region 506 increases. Alternatively, the optical sensor 404 can measure the number of darker pixels that form the analysis region 506.

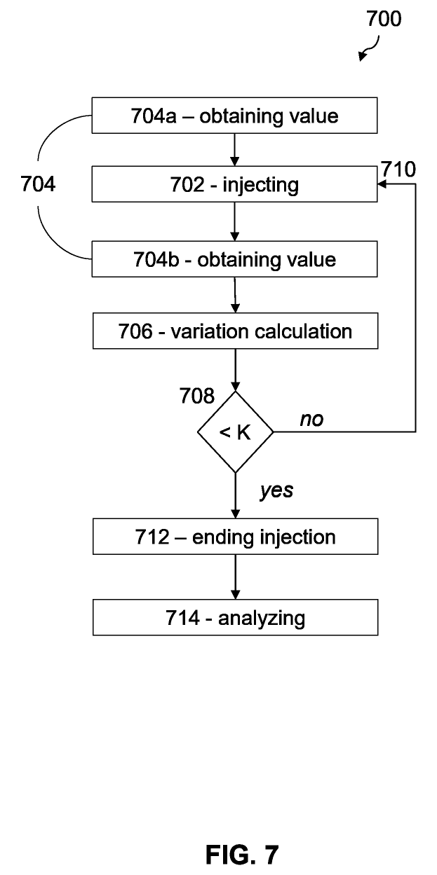

An automatic injection control method 700 is shown in FIG. 7. The injector 310 is controlled by the control circuitry 800 described in FIG. 8 below.

In step 702, injector 310 injects liquid onto absorbent material 504 of test support 312. Typically, control circuitry 800 instructs injector 310 to inject. In step 706, the control circuitry 800 calculates a change in a physical value relating to a physical property of the control region 600. The physical value is obtained using the optical sensor 404, for example by image processing (pixel processing). To calculate a variation in the value, at least two values of the physical property are obtained at intervals in time, with liquid injection between the obtaining of the two values. To do this, the calculation of the variation 706 is preceded by obtaining 704 of two values of the physical property spaced apart in time (for example, T0 and T1, or T1 and T2, in FIG. 6) with the injection of liquid 702 between two obtainings 704a, 704b. In particular, the method 700 may include an obtaining step 704a of a value of the physical property before the start of injection 702 and therefore before the test support is saturated (i.e., when it is dry). Another obtaining step 704b takes place after liquid has been injected. Alternatively, the obtaining steps for a value 704 begin after the start of injection 702. In some implementations, the values are obtained periodically at a fixed sampling interval (e.g., once every 0.1–2 seconds) during injection.

For step 706, the variation may be obtained by a derivative calculation, such as a discrete derivative, or more simply as a percentage of absolute variation.

Then, in step 708, the variation in the value calculated in step 706 is compared to a threshold K. In step 710, in response to determining that the variation is greater than or equal to threshold K, control circuitry 800 continues or resumes injection from step 702. In step 712, in response to determining that the variation (i.e., the variation in the value of the physical property) is less than threshold K, control circuitry 800 stops injection. In an embodiment, threshold K is less than 5%. In some embodiments, K is selected to account for sensor noise and normal variability in illumination, such that K represents a minimum meaningful change in the physical property indicative of continued wetting rather than measurement jitter.

The automatic injection control method 700 therefore includes a control loop that continues the injection until the variations in the physical property of the control region 600 are low, which indicates saturation of the imbibition and therefore means that the injection can be terminated. In other words, injection is maintained while the monitored property is changing and is terminated when the monitored property stabilizes, thereby providing a saturation-based stopping criterion.

Depending on whether the injection is continuous or intermittent, the value can be obtained when there is no injection or simultaneously with an injection. For example, in intermittent operation the value may be measured during pauses between pulses, whereas in continuous operation the value may be measured while injection continues.

In the intermittent mode of operation, the injection is performed in intermittent injection sequences and a value of the parameter is obtained before an injection sequence and a value of the parameter is obtained after the injection sequence. The method thus comprises the following steps: obtaining a value V1 of the parameter, injecting liquid #1 onto the test support 312, obtaining a value V2 of the parameter, calculating the variation Δ(V1, V2), then comparing it with the threshold, and if Δ(V1, V2) > K, then injecting liquid #2 onto the test support 312, obtaining a value V3 of the parameter, calculating the variation Δ(V2, V3), then comparing it with the threshold, etc. In general terms, the method thus comprises: obtaining a value Vk for the parameter, injection #n, obtaining a value Vk+1, calculating the variation Δ(Vk, Vk+1), then comparing with the threshold K, etc. In some embodiments, each injection sequence is a pulse having a predetermined duration and/or volume, and the obtaining steps occur after a predetermined settling time following each pulse to reduce motion- or droplet-induced optical artifacts.

For example, in the continuous mode of operation, injection is performed continuously and the variation is calculated continuously, for example using two moving values spaced at a fixed interval in time (e.g., a few seconds). In one example, V(i) is computed from a moving average of images collected over a first time window and compared to V(i−m) computed from a second time window offset by the fixed interval.

The number of times that circuitry 800 performs a variation calculation is at least two, or even five. In other words, the control loop is performed at least two, or even five times. In the case of continuous injection, the calculation is performed for at least 5 seconds. In some embodiments, the control loop continues until (i) the variation remains below threshold K for N consecutive calculations (e.g., N=2–5) and/or (ii) a maximum injection time or maximum injected volume is reached as a safety limit.

### Robustness of the method

Taking into account the absence (or low level) of variation in the value of the physical parameter makes it possible to obtain a generically applicable method, without the test support providing specific means of controlling imbibition.

In addition, the method is robust to aging since it eliminates the need for an absolute criterion in relation to physical properties (such as a particular color or other characteristic). In the case of a urine analysis device, the test supports 312 may be required to remain in a humid environment for several months, and even if they are arranged in a sealed cavity (see WO documents for more details), the components of the test supports 312 may age. Since the comparison is made between two images of the same test support, the method does not require any special or complex calibration.

Similarly, depending on the types and materials of the test supports, the color of the test support 312 may differ slightly from one test support to another, from one batch to another, or from one supplier to another. The method is robust to this type of difference.

The method is also robust to changes in injector flow rate. At the station 200 level, there may be large variations in flow rate due to the presence of air bubbles in the fluidic circuit 300 or due to aging of the pump 306. As the method operates with a feedback loop, it is insensitive to flow loss (see below for more details). If the flow rate doubles in a few days, the injection time will vary, but the result will always be similar.

### Safety principle

In an embodiment, a safety measure is implemented to prevent the station from flooding. For example, the control circuitry 800 may impose one or more injection “failsafe” limits that cap the maximum amount of liquid deliverable during an injection event, independent of the saturation-based stopping criterion described above. In the case of an injector 310 operating by injection sequence, a maximum number of injection sequences is predefined, beyond which the injector 310 stops injecting. Stated differently, the control circuitry 800 counts discrete injection sequences (e.g., pump strokes) and terminates injection when the count reaches a predefined maximum. For example, this number may be thirty pump strokes. Similarly, in the case of continuous injection, a maximum duration is predefined, beyond which the injector 310 stops injection, for example 10 seconds of continuous injection. In some implementations, the safety measure may additionally include a maximum injected volume threshold (e.g., derived from a calibrated volume-per-stroke value or a pump flow rate), such that injection is stopped when any of the limits (sequence count, duration, and/or volume) is reached.

### Test support analysis

After step 712 (imbibition completed), the control circuitry 800 triggers an analysis 714 of the test support 312, in particular of the analysis region 506, to generate analysis data about a parameter of the injected liquid. In an embodiment, this determination is made using the optical sensor 404. For example, the optical sensor 404 captures one or more images of the analysis region 506 (including, in some embodiments, one or more colorimetric pads 506a, 506b), and the control circuitry 800 performs image processing to determine a measured optical response (e.g., a color value, intensity, reflectance/transmittance proxy, or other optical metric) indicative of the parameter being tested. The optical sensor is therefore used both for optical analysis and for injection control. Accordingly, a single sensing modality (optical sensing) may be leveraged both to (i) determine when the test support 312 has reached a suitable wetting/saturation condition and (ii) read out the assay response after imbibition. The injection is therefore controlled without additional means, i.e., without any specific component dedicated to injection control. This can reduce component count, simplify assembly, and improve reliability by avoiding a separate dedicated flow or wetness sensor for injection control. By using a sensor 404 that sees the entire surface of the test support 312, the same optical sensor 404 can be used to control the injection and analyze the test support 312. In some embodiments, the optical sensor 404 provides a field of view that includes at least the injection region 510, the control region 600, and the analysis region 506, enabling both the saturation determination and the subsequent assay readout without repositioning the sensor.

### Control architecture and control circuitry

FIG. 8 schematically illustrates a station 200 with control circuitry 800. The control circuitry 800 comprises a processor 802, a memory 804 (RAM or ROM, for example permanent) and an i/o interface 806 for exchanging data. In some embodiments, the processor 802 executes firmware and/or software instructions stored in memory 804 to implement the injection control loop (e.g., method 700), the safety limits described above, and the post-injection analysis 714.

The memory 804 can store programs that can be executed by the processor 802. These programs then implement a method as described above. The memory 804 may additionally store configuration parameters (e.g., threshold K, sampling intervals, maximum injection duration, maximum stroke count, calibration values, and/or image-processing parameters) and/or lookup tables used for converting measured optical responses to reported analysis results.

Station 200 also includes a battery 808 configured to supply power to the electrical or electronic components of station 200. In some embodiments, the control circuitry 800 manages power consumption by selectively powering the pump 306, motors 810/812, light source 402, and/or wireless module 816 only when needed.

The control circuitry 800 can, in particular, control the pump 306 to drive the injection, as well as a motor 810 to move the cartridge 202 in the station 200, and a motor 812 to move the injection end 308 into the injection position (see WO documents). The control circuitry 800 can, in particular, exchange information with the fluid presence sensors 314, 316. For example, the control circuitry 800 may inhibit initiation of injection unless the fluid presence sensors 314, 316 indicate that liquid is available, and/or may stop injection and generate an error condition if liquid presence is not detected during injection.

Station 200 may also include a wireless communication module 816 (e.g., a BLUETOOTH® (A short-range wireless technology taht enables two devices to connect directly without requiring supporting network infrastructure such as a wireless router or access point), BLUETOOTH LOW ENERGY (it is optimized for low power consumption and mainly used for applications that are constrained by battery life, and/or Wi-Fi ) connected to control circuitry 800. The module 816 allows information to be exchanged (transmission and reception) via a communication network 818 with a mobile terminal 820 (e.g., a smartphone) and/or a remote server 822. The communication network 818 may be wireless and/or wired and/or a combination of both. The analysis data can thus be sent to the server 822 and then transmitted to the mobile terminal 820 (or communicated directly to the mobile terminal 820, which then transmits it to the server). Conversely, the station 200 can receive updates from the remote server 822 or the mobile terminal 820, such as a computer program as described above. In some embodiments, transmitted data may include timestamps, device identifiers, quality-control metrics (e.g., whether injection terminated by saturation or by a failsafe limit), and/or processed/normalized assay values.

### Variations

The present description has presented an injector 310 with an injection end 308 and a pump 306. However, the injector may take other forms adapted to the device in which it is used. The injector, in general, means a system for delivering a liquid in a controlled manner. For example, the injector 310 may comprise a peristaltic pump, a diaphragm pump, a syringe-based actuator, a microfluidic dosing pump, a piezoelectric dispenser, a solenoid valve coupled to a pressurized reservoir, and/or another metering mechanism configured to deliver discrete volumes and/or continuous flow under control of the control circuitry 800. In such variations, the automatic injection control method may be applied by monitoring, via the optical sensor 404, a selected control region 600 and terminating delivery when the monitored physical property stabilizes as described herein.

In an embodiment of the disclosure, there is provided a method of controlling delivery of urine to a test support in a urine analysis system, the method comprising: causing an injector to deliver urine to a test support that includes an absorbent material; obtaining, using an optical sensor, a plurality of optical measurements associated with a control region of the test support at different times during said delivery; determining, by control circuitry based on the plurality of optical measurements, successive values of a monitored metric representative of a physical property of the control region; computing, by the control circuitry, a change value indicative of an amount of change of the monitored metric between the successive values; comparing the change value to a threshold; and controlling the injector based on the comparing, including stopping the delivery of urine in response to determining that the change value is below the threshold, thereby indicating saturation of the control region.

In an embodiment, the monitored metric comprises a color value of the control region determined from image data captured by the optical sensor. In an embodiment, the color value comprises at least one channel value of an RGB representation of the image data. In an embodiment, the physical property comprises a transparency of the absorbent material in the control region, and the monitored metric is representative of a light intensity transmitted through or reflected from the control region. In an embodiment, the control region comprises an extremal or end region of the absorbent material positioned downstream of an injection region along a direction of urine spreading through the absorbent material. In an embodiment, the physical property comprises a wetting-induced change in appearance of the extremal region due to increasing saturation of the absorbent material. In an embodiment, the control region comprises at least a portion of an analysis region that includes a colorimetric pad configured to change color in response to contact with urine. In an embodiment, the physical property comprises an area associated with wetting of the test support, and wherein the monitored metric comprises a pixel-count value derived from the optical measurements. In an embodiment, the pixel-count value comprises a number of saturated pixels adjacent the analysis region that decreases as an area of the analysis region increases due to absorption of urine. In an embodiment, the pixel-count value comprises a number of darker pixels corresponding to the analysis region. In an embodiment, computing the change value comprises computing a discrete derivative of the monitored metric. In an embodiment, computing the change value comprises computing a percent change between successive values of the monitored metric. In an embodiment, determining the successive values of the monitored metric comprises computing a spatial average over at least a portion of the control region in image data captured by the optical sensor. In an embodiment, the method further comprises temporally averaging the spatial average over a plurality of images to obtain at least one of the successive values of the monitored metric. In an embodiment, causing the injector to deliver urine comprises delivering urine in intermittent injection sequences, and obtaining the plurality of optical measurements comprises obtaining a first optical measurement before a given injection sequence and obtaining a second optical measurement after the given injection sequence. In an embodiment, controlling the injector comprises performing a control loop that repeats the intermittent injection sequences until the change value is below the threshold. In an embodiment, the injector performs a first injection sequence, the optical sensor obtains a first value of the monitored metric, the injector performs a second injection sequence responsive to the change value being greater than or equal to the threshold, and the optical sensor obtains a second value of the monitored metric after the second injection sequence. In an embodiment, causing the injector to deliver urine comprises delivering urine continuously, and obtaining the plurality of optical measurements comprises obtaining optical measurements at a fixed sampling interval during the continuous delivery. In an embodiment, computing the change value comprises computing the change value using two values of the monitored metric separated by a fixed time interval. In an embodiment, the method further comprises stopping the delivery of urine upon reaching a safety limit comprising at least one of (i) a maximum number of injection sequences and (ii) a maximum injection duration, independent of the threshold comparison. In an embodiment, the method further comprises, after stopping the delivery of urine, analyzing an analysis region of the test support using the optical sensor to generate analysis data indicative of at least one parameter of the urine. In an embodiment, the threshold is less than 5%. In an embodiment, causing the injector to deliver urine comprises delivering urine in intermittent injection sequences, and obtaining the plurality of optical measurements comprises obtaining a first optical measurement before a given injection sequence and obtaining a second optical measurement after the given injection sequence.

The articles "a" and "an" may be employed in connection with various elements and components of compositions, processes or structures described herein. This is merely for convenience and to give a general sense of the compositions, processes or structures. Such a description includes "one or at least one" of the elements or components. Moreover, as used herein, the singular articles also include a description of a plurality of elements or components, unless it is apparent from a specific context that the plural is excluded.

As used herein in the specification and in the claims, the phrase “at least one”, in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

A person skilled in the art will readily appreciate that various features, elements, parameters disclosed in the description may be modified and that various embodiments disclosed may be combined without departing from the scope of the invention. For example, various aspects of the present disclosure may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Having described above several aspects of at least one embodiment, it is to be appreciated various alterations, modifications, and improvements will readily occur to those skilled in the art. Such alterations, modifications, and improvements are intended to be aspects of this disclosure. Accordingly, the foregoing description and drawings are by way of example only.

1. An injection control method for a liquid on a test support, the test support comprising an absorbent material adapted to absorb the liquid and a reagent adapted to react with at least one component present in the liquid, the method being implemented using an injector and an optical sensor, the method comprising:
injecting the liquid onto the absorbent material of the test support by the injector,
calculating a change in a value of a physical property using the optical sensor of a control region of the test support during the injection,
in response to determining that the variation is less than a threshold, ending the injection.

2. The injection control method according to claim 1, wherein, in response to determining that the variation is greater than the threshold, continuing the injection of liquid onto the test support.

3. The injection control method according to claim 1, wherein:
injection is performed in intermittent injection sequences, and
the variation in the value is calculated from at least one value obtained before an injection sequence and at least one value obtained after the injection sequence.

4. The injection control method according to claim 3, wherein at least three injection sequences take place and at least two calculations of the variation take place before the end of the injection.

5. The injection control method according to claim 1, wherein the injection is continuous and the variation calculation is performed continuously.

6. The injection control method according to claim 1, wherein absorbent material defines a liquid delivery path from an injection region, which receives liquid from the injector, to the control region, wherein the reagents are located along the path between the injection region and the control region.

7. The injection control method according to claim 1, wherein the control region is a region of the absorbent material and the value is a color of the control region.

8. The injection control method according to claim 1, wherein the control region is a colorimetric pad of the test support.

9. The injection control method according to claim 8, wherein the physical property is a color of the colorimetric pad (506a, 506b).

10. The injection control method according to claim 8, wherein the physical property is a dimension of the colorimetric pad (506a, 506b).

11. The injection control method according to claim 1, further comprising:
in response to the end of injection, analyzing the test support using the optical sensor.

12. A test support analysis station, comprising an injector and an optical sensor, the analysis station being configured to implement a method according to claim 1.

13. A station according to claim 12, wherein the optical sensor is configured to view the entire test support.

14. A urine analysis device comprising a station according to claim 12, and a test support, wherein the test support comprises an absorbent material adapted to absorb liquid and a reagent adapted to react with at least one component present in the liquid.

15. A non-transitory computer program product comprising instructions configured to implement a method according to claim 1 when the instructions are executed by a processor.

16. A method of controlling delivery of urine to a test support in a urine analysis system, the method comprising:
causing an injector to deliver urine to a test support that includes an absorbent material;
obtaining, using an optical sensor, a plurality of optical measurements associated with a control region of the test support at different times during said delivery;
determining, by control circuitry based on the plurality of optical measurements, successive values of a monitored metric representative of a physical property of the control region;
computing, by the control circuitry, a change value indicative of an amount of change of the monitored metric between the successive values;
comparing the change value to a threshold; and
controlling the injector based on the comparing, including stopping the delivery of urine in response to determining that the change value is below the threshold, thereby indicating saturation of the control region.

17. The method of claim 16, wherein the monitored metric comprises a color value of the control region determined from image data captured by the optical sensor.

18. The method of claim 17, wherein the color value comprises at least one channel value of an RGB representation of the image data.

19. The method of claim 16, wherein the physical property comprises a transparency of the absorbent material in the control region, and wherein the monitored metric is representative of a light intensity transmitted through or reflected from the control region.

20. The method of claim 16, wherein causing the injector to deliver urine comprises delivering urine in intermittent injection sequences, and wherein obtaining the plurality of optical measurements comprises obtaining a first optical measurement before a given injection sequence and obtaining a second optical measurement after the given injection sequence.
